(19) United States
(12) Patent Application Publication
(10) Pub. No.: US 2023/0390065 A1
(43) Pub. Date: Dec. 7, 2023

(54) INFLATABLE PENILE PROSTHESIS HAVING A PUMP

(71) Applicant: Boston Scientific Scimed, Inc., Maple Grove, MN (US)

(72) Inventors: James Ryan Mujwid, Hudson, WI (US); Abigail Rae Brooks, Saint Louis Park, MN (US); John Anders Bostrom, Minneapolis, MN (US)

(21) Appl. No.: 18/325,489

(22) Filed: May 30, 2023

Related U.S. Application Data
(60) Provisional application No. 63/365,633, filed on Jun. 1, 2022.

(51) Int. Cl. A61F 2/26

(52) U.S. Cl. CPC A61F 2/26; A61F 2250/0013

(57) ABSTRACT

According to an aspect, an implant includes an inflatable member and a pump assembly. The pump assembly includes a cavity, a valve, and a valve housing. The pump assembly is configured to facilitate a transfer of a fluid from the cavity to the inflatable member. The valve housing is configured to be deformed to allow the fluid to move from the inflatable member to the cavity.

### CROSS-REFERENCE TO RELATED APPLICATION

This application claims priority to U.S. Provisional Patent Application No. 63/365,633, filed on Jun. 1, 2022, entitled “INFLATABLE PENILE PROSTHESIS HAVING A PUMP”, the disclosure of which is incorporated by reference herein in its entirety.

### TECHNICAL FIELD

This disclosure relates generally to bodily implants and more specifically to bodily implants, such as penile prostheses that include a pump.

### BACKGROUND

One treatment for male erectile dysfunction is the implantation of a penile prosthesis that mechanically erects the penis. Additionally, some phalloplasty patients may receive a penile prosthesis. Some existing penile prostheses include inflatable cylinders or members that can be inflated or deflated using a pump mechanism. In some existing devices, the inflatable cylinder or member requires a relatively large amount of force to inflate. Additionally, in some existing devices, the pump mechanism may require many sequential squeezes or activations to inflate the cylinder or member. Furthermore, in some existing devices, the pump mechanism may be small or difficult to locate and squeeze or both.

### SUMMARY

According to an aspect, an implant includes an inflatable member and a pump assembly. The pump assembly includes a cavity, a valve, and a valve housing. The pump assembly is configured to facilitate a transfer of a fluid from the cavity to the inflatable member. The valve housing is configured to be deformed to allow the fluid to move from the inflatable member to the cavity.

In some embodiments, the cavity includes a first portion, a second portion, and a transition portion being disposed between the first portion and the second portion. In some embodiments, the cavity includes a first portion, a second portion, and a transition portion being disposed between the first portion and the second portion, the first portion having a first width, the transition portion having a second width, the second width being smaller than the first width. In some embodiments, the cavity includes a first portion, a second portion, and a transition portion being disposed between the first portion and the second portion, the first portion having a first width, the second portion having a second width, the transition portion having a third width, the third width begin smaller than the first width, the third width being smaller than the second width.

In some embodiments, the cavity includes a first portion, a second portion, and a transition portion being disposed between the first portion and the second portion, the first portion having a first height, the transition portion having a second height, the second height being smaller than the first height. In some embodiments, the cavity includes a first portion, a second portion, and a transition portion being disposed between the first portion and the second portion, the first portion having a first height, the second portion having a second height, the transition portion having a third height, the third height being smaller than the first height, the third height being smaller than the second height.

In some embodiments, the cavity includes a first portion, a second portion, and a transition portion being disposed between the first portion and the second portion, the first portion having a first width and a first height, the transition portion having a second width and a second height, the second height being smaller than the first height, the second width being smaller than the first width.

In some embodiments, the valve includes a first portion, a second portion, and a middle portion being disposed between the first portion and the second portion. In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width. In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a portion of the valve housing to form a seal.

In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal.

In some embodiments, the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal, the seat surface being configured to be moved from the seat portion of the valve housing when the valve housing is deformed.

In some embodiments, the valve housing is configured to be deformed when the valve housing is compressed. In some embodiments, the valve housing is configured to be deformed when the valve housing is compressed by a user. In some embodiments, the valve housing defines a compartment, the valve being disposed within the compartment, the compartment having a first portion, a second portion, and a third portion disposed between the first portion and the second portion, the first portion having a first width, the second portion having a second width, the third portion having a third width, the third width being smaller than the first width, the third width being smaller than the second width.

According to another aspect, a method includes inflating an inflatable member of a bodily implant, the inflating including squeezing a pump assembly of the bodily implant; and deflating the inflatable member, the deflating including deforming a valve housing to unseat a valve of the bodily implant.

In some embodiments, the inflating includes squeezing the pump assembly of the bodily implant a single time. In some embodiments, the inflating includes squeezing the pump assembly once and only once. In some embodiments, the pump assembly includes a cavity having a first portion, a second portion, and a transition portion being disposed between the first portion and the second portion, the first portion having a first width, the transition portion having a second width, the second width being smaller than the first width, the inflating includes squeezing the first portion of the cavity and squeezing the second portion of the cavity.

In some embodiments, the pump assembly includes a cavity having a first portion, a second portion, and a transition portion being disposed between the first portion and the second portion, the first portion having a first width and a first height, the transition portion having a second width and a second height, the second height being smaller than the first height, the second width being smaller than the first width, the inflating includes squeezing the first portion of the cavity and squeezing the second portion of the cavity.

In some embodiments, the deflating includes squeezing a portion of the valve housing.

### BRIEF DESCRIPTION OF THE DRAWINGS

FIG. 1 schematically illustrates a penile prosthesis according to an embodiment.

FIG. 2 illustrates a penile prosthesis according to another embodiment.

FIG. 3 illustrates a portion of the penile prosthesis of FIG. 2.

FIG. 4 illustrates a portion of the penile prosthesis according to another embodiment.

FIG. 5 is a flow chart of a method according to an embodiment.

### DETAILED DESCRIPTION

Detailed embodiments are disclosed herein. However, it is understood that the disclosed embodiments are merely examples, which may be embodied in various forms. Therefore, specific structural and functional details disclosed herein are not to be interpreted as limiting, but merely as a basis for the claims and as a representative basis for teaching one skilled in the art to variously employ the embodiments in virtually any appropriately detailed structure. Further, the terms and phrases used herein are not intended to be limiting, but to provide an understandable description of the present disclosure.

The terms “a” or “an,” as used herein, are defined as one or more than one. The term “another,” as used herein, is defined as at least a second or more. The terms “including” and/or “having”, as used herein, are defined as comprising (i.e., open transition). The term “coupled” or “moveably coupled,” as used herein, is defined as connected, although not necessarily directly and mechanically.

In general, the embodiments are directed to medical devices such as penile prostheses or other bodily implants. The term patient or user may hereafter be used for a person who benefits from the medical device or the methods disclosed in the present disclosure. For example, the patient can be a person whose body is implanted with the medical device or the method disclosed for operating the medical device by the present disclosure. For example, in some embodiments, the patient may be a human male, a human female, or any other mammal.

The embodiments discussed herein may improve the performance of an inflatable member of the device. For example, the inflatable member may have improved stiffness or rigidity, improved reliability, or improved deflation or inflation times. In some embodiments, inflating the inflatable member may be facilitated by requiring less force, less pressure, or less fluid transfer to inflate the inflatable member. Additionally, in some embodiments, the inflatable member may be inflated with a single squeeze of the pump.

The embodiments may include an inflatable penile prosthesis having a pump assembly and an inflatable member. The inflatable member may be implanted into the corpus cavernosae of a patient or user and the pump assembly may be implanted in the scrotum.

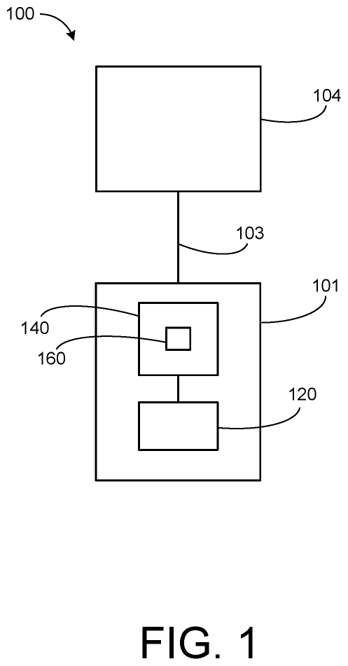

FIG. 1 schematically illustrates an inflatable penile prosthesis 100 according to an aspect. The inflatable penile prosthesis 100 includes a cylinder or inflatable member 104 and a pump assembly 101 configured to transfer fluid to and from the inflatable member 104. In some examples, the inflatable member 104 may be implanted into the corpus cavernosae of the user and the pump assembly 101 may be implanted in the scrotum of the user.

The inflatable member 104 may be capable of expanding upon the injection of fluid into a cavity of the inflatable member 104. For instance, upon injection of the fluid into the inflatable member 104, the inflatable member 104 may increase its length and/or width, as well as increase its rigidity. In some examples, the inflatable member 104 may include a pair of cylinders or at least two cylinders, e.g., a first cylinder member and a second cylinder member. The volumetric capacity of the inflatable member 104 may depend on the size of the cylinders. In some examples, the volume of fluid in each cylinder may vary from about 10 milliliters in smaller cylinders and to about milliliters in larger sizes. In some examples, the first cylinder member may be larger than the second cylinder member. In other examples, the first cylinder member may be the same size as the second cylinder member.

The inflatable penile prosthesis 100 includes a conduit connector 103. The conduit connector 103 may define a lumen configured to transfer the fluid to and from the pump assembly 101. The conduit connector 103 may be coupled to the pump assembly 101 and to the inflatable member 104. The conduit connector 103 may include a single or multiple tube members for transferring the fluid between the pump assembly 101 and the inflatable member 104.

The pump assembly 101 may include a cavity or a cavity portion 120. The cavity or cavity portion 120 may be configured to retain fluid that is transferred to the inflatable member 104 to place the inflatable member in an inflated configuration. The cavity portion 120 may include more than one portion and may be sized and configured to be gripped between a palm of a user and fingers of the user.

The pump assembly 101 also includes a valve housing 140 and a valve 160. The valve housing 140 is operatively coupled to the cavity 120. In some embodiments, the valve housing 140 is fluidically coupled to the cavity 120. The valve 160 is configured to regulate the flow of fluid between the inflatable member 104 and the cavity 120.

In some embodiments, the valve housing 140 is configured to be deformed to allow the fluid to flow from the inflatable member 104 to the cavity 120. For example, in some embodiments, the valve housing 140 is formed of a material that may be deformed upon a compression force. In some embodiments, a user may squeeze or otherwise compress the valve housing 140 to deform the valve housing 140. In some embodiments, the deformation of the valve housing 140 causes the valve 160 to become unseated and allow the fluid in the inflatable member 104 to flow to the cavity 120 to place the inflatable member 104 in a deflated configuration.

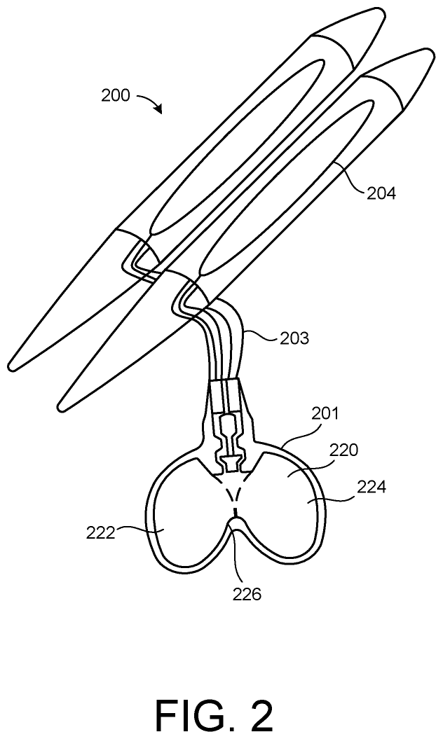

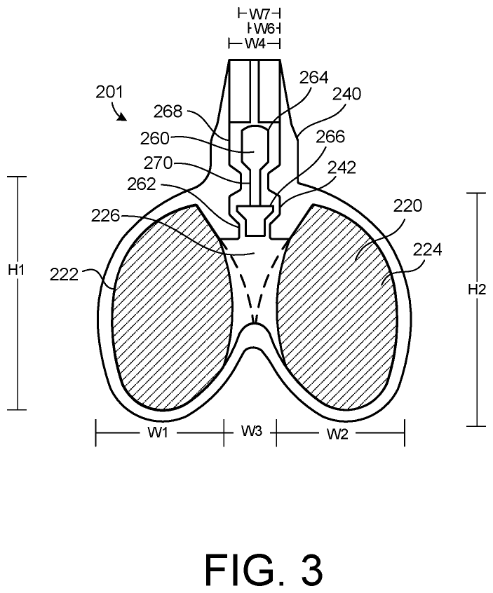

FIG. 2 illustrates a penile prosthesis 200 according to an aspect. FIG. 3 illustrates a pump assembly 201 of the penile prosthesis 200.

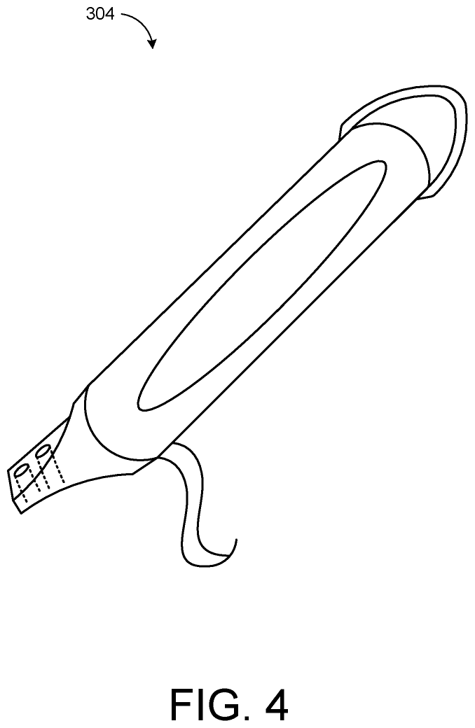

The inflatable penile prosthesis 200 includes an inflatable member 204 and a pump assembly 201 configured to transfer fluid to and from the inflatable member 204. In the illustrated embodiment, the inflatable member 204 includes two or a pair of inflatable cylinders. As illustrated in FIG. 4, in other embodiments, an inflatable member 304 may include a single inflatable cylinder.

The inflatable member 204 may be implanted into the corpus cavernosae of the user or patient and the pump assembly 201 may be implanted in the scrotum of the user or patient.

The inflatable member 204 may be capable of expanding upon the injection of fluid into a cavity of the inflatable member 204. For instance, upon injection of the fluid into the inflatable member 204, the inflatable member 204 may increase its length and/or width, as well as increase its rigidity. In some examples, the inflatable member 204 may include a pair of cylinders or at least two cylinders, e.g., a first cylinder member and a second cylinder member. The volumetric capacity of the inflatable member 204 may depend on the size of the cylinders. In some examples, the volume of fluid in each cylinder may vary from about 10 milliliters in smaller cylinders and to about milliliters in larger sizes. In some examples, one cylinder member may be larger than the other cylinder member. In other examples, the first cylinder member may be the same size as the second cylinder member.

The inflatable penile prosthesis 200 includes a conduit connector 203. The conduit connector 203 defines a lumen configured to transfer the fluid to and from the pump assembly 201 and the inflatable member 204. The conduit connector 203 is coupled to the pump assembly 201 and to the inflatable member 204. The conduit connector 203 may include a single or multiple tube members for transferring the fluid between the pump assembly 201 and the inflatable member 204.

The pump assembly 201 includes a cavity or a cavity portion 220. The cavity or cavity portion 220 is configured to retain fluid that is transferred to the inflatable member 204 to place the inflatable member in an inflated configuration. The cavity portion 220 is sized such that it may be gripped between a palm of a user and fingers of the user and squeezed by the user.

In some embodiments, a single squeeze (one and only one squeeze) of the cavity portion 220 by the user will cause enough fluid to flow from the cavity portion, past the valve 160, and into the inflatable member 204 to allow the inflatable member 204 to inflate from a deflated configuration. In other embodiments, a user may squeeze the cavity portion 220 more than once to inflate the inflatable member 204.

In the illustrated embodiment, the cavity or cavity portion 220 includes a first portion 222, a second portion 224, and a transition portion 226. The first portion 222, the second portion 224, and the transition portion 226 are fluidically coupled and the transition portion 226 is disposed between the first portion 222 and the second portion 224.

The first portion 222 of the cavity portion 220 has a first width W1. The second portion 224 of the cavity portion 220 has a second width W2. The transition portion 226 has a third width W3. The width W1 of the first portion 222 is substantially the same as the width W2 of the second portion 224. The width W3 of the transition portion 226 is smaller than the width W1 of the first portion 222. The width W3 of the transition portion 226 is smaller than the width W2 of the second portion 224.

The first portion 222 of the cavity portion 220 has a first height H1. The second portion 224 of the cavity portion 220 has a second height H2. The transition portion 226 has a third height H3. The height H3 of the transition portion 226 is smaller than the height H1 of the first portion 222. The height H3 of the transition portion 226 is smaller than the height H2 of the second portion 224.

The pump assembly 201 also includes a valve housing 240 and a valve 260. The valve housing 240 is operatively coupled to the cavity 220. In the illustrated embodiment, the valve housing 240 is fluidically coupled to the cavity 220. The valve 260 is configured to regulate the flow of fluid between the inflatable member 204 and the cavity 220.

The valve 260 includes a seat portion or surface 262 to that is configured to engage a seat or sealing portion 242 of the valve housing 240. In the illustrated embodiment, the valve 260 is biased towards the seat or sealing portion 242 of the valve housing 240. Specifically, in the illustrated embodiment, a spring or other biasing member 264 is disposed such that it biases the seat or sealing portion 262 of the valve 260 towards the seat or sealing portion 242 of the valve housing 240.

In the illustrated embodiment, the valve 260 includes a first end portion 266, a second end portion 268, and a middle portion 270 disposed between the first end portion 266 and the second end portion 268. The first end portion 266 has a width W4. The second end portion 268 has a width W5. The middle portion 270 has a width W6. The width W6 of the middle portion 270 is smaller than the width W4 of the first end portion W4. The width W6 of the middle portion 270 is smaller than the width W5 of the second end portion 268.

The valve housing 240 defines a cavity 244. The valve 260 is disposed within the cavity 244. The cavity 244 includes a first portion 246, a second portion 248, and a middle portion 250. The first portion 246 of the cavity 244 has a width W7. The second portion 248 of the cavity 244 has a width W8. The middle portion 250 has a width W9. The width W9 of the middle portion 250 is smaller than the width W7 of the first portion 246. The width W9 of the middle portion 250 is smaller than the width W8 of the second portion 248.

The first end portion 266 of the valve 260 is disposed within the first portion 246 of the cavity 244. The second end portion 268 of the valve 260 is disposed within the second portion 248 of the cavity 244. The middle portion 270 of the valve 260 is disposed within the middle portion 250 of the cavity 244.

The valve housing 240 is configured to be deformed to allow the fluid to flow from the inflatable member 204 to the cavity 220. For example, in some embodiments, the valve housing 240 is formed of a material that may be deformed upon a compression force. In some embodiments, a user may squeeze or otherwise compress the valve housing 240 to deform the valve housing 240. In some embodiments, the deformation of the valve housing 240 causes the valve 260 to become unseated and allow the fluid in the inflatable member 204 to flow to the cavity 220 to place the inflatable member 204 in a deflated configuration.

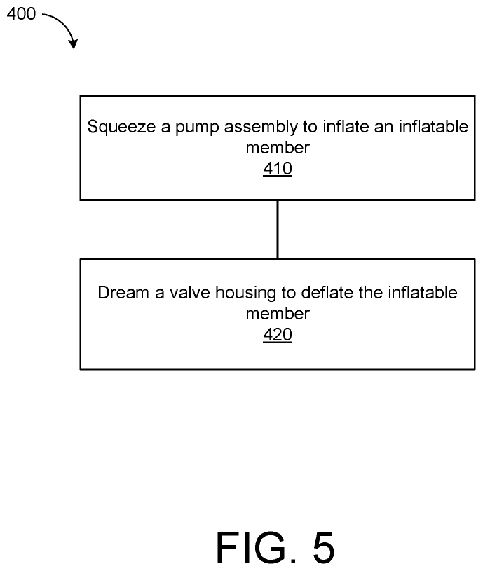

FIG. 5 is a flow chart of a method 400 according to an embodiment. At 410 a pump assembly is squeezed to inflate an inflatable member of the bodily implant. In some embodiments, a cavity portion of the pump assembly is squeezed. In some embodiments, the pump assembly is squeezed once and only once (a single squeeze) to inflate the inflatable member. In some embodiments, the squeezing of the pump assembly causes fluid located in a cavity of the pump assembly to move from the cavity of the pump assembly to the inflatable member.

At 420, a valve housing is deformed to deflate the inflatable member. In some embodiments, the deformation of the valve housing allows fluid in the inflatable member to move from the inflatable member, past a valve member, and into the cavity of the pump assembly. In some embodiments, the deformation of the valve housing causes the valve to move from a seated or sealed position.

In other embodiments, a user may fold the inflatable member such as fold the inflatable member in half to deflate the inflatable member. In some embodiments, the pressure of the folding of the inflatable member causes the valve housing to deform and allow fluid to pass to the cavity of the pump assembly. In other embodiments, the valve housing may define slots or grooves that allow the fluid to pass the valve when the pressure within the inflatable member is increased, such as by folding or bending the inflatable member.

While certain features of the described implementations have been illustrated as described herein, many modifications, substitutions, changes and equivalents will now occur to those skilled in the art. It is, therefore, to be understood that the appended claims are intended to cover all such modifications and changes as fall within the scope of the embodiments.

What is claimed is:

1. An implant, comprising:
an inflatable member; and
a pump assembly having a cavity, a valve, and a valve housing, the pump assembly being configured to facilitate a transfer of a fluid from the cavity to the inflatable member, the valve housing being configured to be deformed to allow the fluid to move from the inflatable member to the cavity.

2. The implant of claim 1, wherein the cavity includes a first portion, a second portion, and a transition portion being disposed between the first portion and the second portion.

3. The implant of claim 1, wherein the cavity includes a first portion, a second portion, and a transition portion being disposed between the first portion and the second portion, the first portion having a first width, the transition portion having a second width, the second width being smaller than the first width.

4. The implant of claim 1, wherein the cavity includes a first portion, a second portion, and a transition portion being disposed between the first portion and the second portion, the first portion having a first width, the second portion having a second width, the transition portion having a third width, the third width begin smaller than the first width, the third width being smaller than the second width.

5. The implant of claim 1, wherein the cavity includes a first portion, a second portion, and a transition portion being disposed between the first portion and the second portion, the first portion having a first height, the transition portion having a second height, the second height being smaller than the first height.

6. The implant of claim 1, wherein the cavity includes a first portion, a second portion, and a transition portion being disposed between the first portion and the second portion, the first portion having a first height, the second portion having a second height, the transition portion having a third height, the third height being smaller than the first height, the third height being smaller than the second height.

7. The implant of claim 1, wherein the cavity includes a first portion, a second portion, and a transition portion being disposed between the first portion and the second portion, the first portion having a first width and a first height, the transition portion having a second width and a second height, the second height being smaller than the first height, the second width being smaller than the first width.

8. The implant of claim 1, wherein the valve includes a first portion, a second portion, and a middle portion being disposed between the first portion and the second portion.

9. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width.

10. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a portion of the valve housing to form a seal.

11. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal.

12. The implant of claim 1, wherein the valve includes a first portion, a second portion, and a middle portion being disposed between the first portion and the second portion, the first portion having a first width, the second portion having a second width, the middle portion having a third width, the third width being smaller than the first width, the third width being smaller than the second width, the first portion having a seat surface and being configured to engage a seat portion of the valve housing to form a seal, the pump assembly including a bias member configured to bias the seat surface towards the seat portion of the valve housing to form a seal, the seat surface being configured to be moved from the seat portion of the valve housing when the valve housing is deformed.

13. The implant of claim 1, wherein the valve housing is configured to be deformed when the valve housing is compressed.

14. The implant of claim 1, wherein the valve housing is configured to be deformed when the valve housing is compressed by a user.

15. The implant of claim 1, wherein the valve housing defines a compartment, the valve being disposed within the compartment, the compartment having a first portion, a second portion, and a third portion disposed between the first portion and the second portion, the first portion having a first width, the second portion having a second width, the third portion having a third width, the third width being smaller than the first width, the third width being smaller than the second width.

16. A method, comprising:
inflating an inflatable member of a bodily implant, the inflating including squeezing a pump assembly of the bodily implant; and
deflating the inflatable member, the deflating including deforming a valve housing to unseat a valve of the bodily implant.

17. The method of claim 16, wherein the inflating includes squeezing the pump assembly of the bodily implant a single time.

18. The method of claim 16, wherein the inflating includes squeezing the pump assembly once and only once.

19. The method of claim 16, wherein the pump assembly includes a cavity having a first portion, a second portion, and a transition portion being disposed between the first portion and the second portion, the first portion having a first width, the transition portion having a second width, the second width being smaller than the first width, the inflating includes squeezing the first portion of the cavity and squeezing the second portion of the cavity.

20. The method of claim 19, wherein the pump assembly includes a cavity having a first portion, a second portion, and a transition portion being disposed between the first portion and the second portion, the first portion having a first width and a first height, the transition portion having a second width and a second height, the second height being smaller than the first height, the second width being smaller than the first width, the inflating includes squeezing the first portion of the cavity and squeezing the second portion of the cavity.
